(19) United States
(12) Patent Application Publication
(10) Pub. No.: US 2024/0277974 A1
(43) Pub. Date: Aug. 22, 2024

(54) Catheter and method for using the same.

(71) Applicant: Jason Oehler, Elmhurst, IL (US)

(72) Inventor: Jason Oehler, Elmhurst, IL (US)

(21) Appl. No.: 18/583,714

(22) Filed: Feb. 21, 2024

Related U.S. Application Data
(60) Provisional application No. 63/447,173, filed on Feb. 21, 2023.

(51) Int. Cl. A61M 25/09; A61M 25/00

(52) U.S. Cl. CPC A61M 25/09; A61M 25/007

(57) ABSTRACT

An elongate body and a hole for slidably passing a wire receiving assembly. The apparatus working with a short and thin cylindrical portion, such as a short wire. The short and thin cylindrical portion, the wire receiving assembly and an open end of the elongate body used to distend or dilate a body channel more easily.

### CROSS-REFERENCE TO RELATED APPLICATIONS

The present application claims the benefit of U.S. Provisional Patent Application No. 63/447,173, filed Feb. 21, 2023, the entirety of which is hereby incorporated by reference.

### BACKGROUND OF THE INVENTION

### Field of the Invention

The inventor noted that the process for inserting a medical device intended to be inserted into a body cavity or channel, a catheter to name one such device, involved several steps of increasingly expanding (dilating) the patient's channel, such as a blood vessel, before the catheter end is inserted. The process requires the use of long wires and is cumbersome.

Because a device like a catheter is long, the wires inserted within the catheter are also long. This makes the end of the catheter distal from a patient, the hub end, crowded with wires and ports. Due to the need for expanding the body channel, like a blood vessel, before inserting the catheter end, insertion of a smaller diameter wire, feeding it through the catheter to the hub end, is a necessary transition to inserting the larger diameter catheter. Handling such long tubes, filaments, wires, or other long and thin cylindrical portions slows down and interferes with the simple process of inserting a tube into a body cavity or channel.

There is a need in the industry for an apparatus that allows users, usually doctors, or nurses, to insert a medical device more easily, such as a catheter into a patient's body channel, and eliminate the need for feeding a long guide, such as a wire, through the catheter to the end of the device distal from the patient, the hub end of a catheter for instance. Such an apparatus would allow users to quickly insert a guide over a wire that has been inserted into the vessel, for example, and easily slip the catheter end over the guide and onto the wire and into a patient's body channel, the blood vessel. I noticed that this short guide wire capable elongate body, a catheter for instance, and method for using a short guide wire capable catheter are not currently available in the industry.

### SUMMARY

The present invention is directed to an apparatus that satisfies this need. I envisioned an elongated body, like that of a catheter, with a hole that is near to the end of the catheter that gets inserted into a patient's body. In such an embodiment, that hole allows passing a dilator, or hollow tube that is common in the industry, through the hole and into the channel of the elongated tube, an inside chamber of the hollow catheter tube for instance.

The dilator extends beyond the end of the novel catheter and receives a wire, which is already inserted into a patient's vessel. The wire would pass through the opening of the short dilator at this end of the catheter. The catheter can then be slid over the dilator and onto the wire and into the vessel. Once the catheter is inserted into the blood vessel, for instance, the short wire can be pulled out from a point near the same end of the catheter, through the dilator, without having to reach back to the catheter hub, which is at the other end of the elongate body.

The improved catheter system is designed to facilitate medical procedures with enhanced precision and efficacy. In one embodiment for example, the catheter system comprises a catheter with at least one first catheter end and an outer surface. The catheter is further equipped with at least one hole extending through its outer surface to a hollow interior, and the at least one first catheter end features an opening for specialized functionality, such as sliding along a wire and an inventive wire receiving assembly.

Integral to the catheter system is the wire receiving assembly comprising at least one tube, wherein the wire receiving assembly is configured to accommodate, such as by receiving, a short wire. The short wire, a vital component of the system that improves previous systems, is slidably located within the at least one tube, facilitating controlled movement of the first catheter end. The wire receiving assembly includes a first end that is removably inserted into the at least one hole of the catheter, enabling traversal of the hollow interior and extension beyond the at least one first catheter end.

I envision that a wire receiving assembly as a dilator. Dilators can have more than one tube for step dilation of a blood vessel. The tubes are most often slid into the wire receiving assembly.

Moreover, the catheter system incorporates features to enhance functionality and usability. The wire receiving assembly acts as a guide for the at least one first catheter end to slide along and over the wire, ensuring smooth and precise movement during medical procedures. Too, the catheter will have an angled portion formed in its outer surface near one of the holes to help when inserting the dilator into the at least one hole of the catheter. This angled portion makes it simpler to slide the end of the dilator into the hole of the catheter. Additionally, provisions are made for optional features such as insertion into a needle port for accessing body parts, and the catheter may include a hub end for further versatility in medical applications.

My invention also contemplates methods for using the improved catheter system. In one method embodiment, the method involves employing a catheter system comprising a catheter with at least one first catheter end, an outer surface, and at least one hole extending through the outer surface to a hollow interior. The at least one first catheter end is equipped with an opening for specialized functionality.

The catheter system further includes a wire receiving assembly comprising at least one tube and a wire preinserted into a blood vessel. The wire is insertable into and slidable within the at least one tube of the wire receiving assembly. The method initiates with the insertion of the wire receiving assembly's first end into the at least one hole of the catheter, facilitating traversal of the hollow interior and extension beyond the at least one first catheter end.

Subsequently, the method involves traversing the hollow interior with the first end of the wire receiving assembly, followed by extending the first end through the opening and beyond the at least one first catheter end. A crucial step entails manipulating the first end to receive the wire, whereby together, the first end and the wire serve to guide the at least one first catheter end over the wire and into the blood vessel.

Optionally, the method may include providing an angled portion formed in the catheter's outer surface adjacent to at least one of the at least one holes, facilitating the guidance of the first end into the hole. Furthermore, the catheter may be optionally provided with a hub end for added convenience and functionality.

In more detail, the catheter and dilator modules are assembled so that the dilator enters a side hole of the catheter and extends beyond the end hole of the catheter. This dilator module is to be placed over the wire which the operator has previously inserted into a body cavity, such as a vein. The catheter then follows the dilator and passes over the wire. Once the dilator and catheter modules are sufficiently into the body cavity, the catheter moves further along, and the dilator and catheter become separated, the dilator naturally backing out through the at least one hole. The wire and dilator will then be held by the user while the catheter is even further advanced into the body cavity. The wire and dilator will be removed entirely, through the side hole, leaving only the catheter in the body cavity.

The method allows the external hub end, the end distal from the patient, of the catheter being placed to be bypassed, this is novel. This method utilizes fewer steps and components, saves time for the operator, and decreases procedure time for the patient and potentially reduces risks of complications.

### BRIEF DESCRIPTION OF THE SEVERAL VIEWS OF THE DRAWINGS

These and other features, aspects and advantages of the present invention will become better understood with regard to the following description, appended claims, and accompanying drawings where:

FIG. 1 shows a perspective view of an embodiment of the present invention.

FIG. 2 shows a perspective view of another embodiment of the present invention shown in portions.

FIG. 3 shows a perspective view of another embodiment of the present invention for use with a human body.

### DESCRIPTION

### Overview

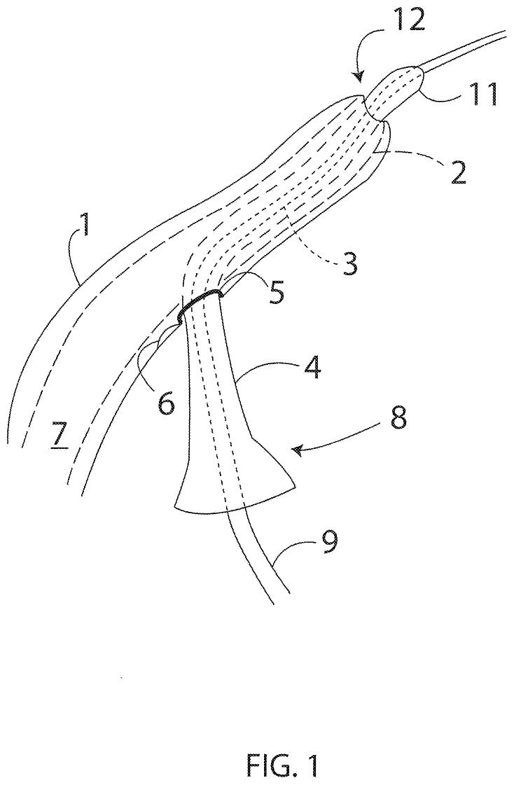

As shown in FIG. 1, a perspective view of an embodiment of the present invention comprises an apparatus that is an improved catheter system. The catheter system has a catheter 1 having an inside passageway, such as a hollow interior 2. The catheter having at least one hole 5 through its outer surface 7. There is an angled portion 6 formed in the outer surface 7. This angled portion acts as a landing spot to guide items into the hole 5.

Shown is a wire 9, best mode is a shorter wire than is customarily used in setting catheters, outside of the hollow interior 2. The wire 3 is also depicted inside the hollow interior 2. A wire receiving assembly 8, such as a dilator that is commonly used in surgical procedures, has at least one tube 4. The at least one tube 4 is shown inserted into the hole 5. The wire 3 is shown passing into the at least one tube 4 and the wire 9 is shown able to travel out the other end of the wire receiving assembly 8. The first end 11 of the wire receiving assembly 4 is shown extended, having been moved in place by a user, beyond the at least one first catheter end 10. A catheter can have many first catheter ends depending on different surgical procedures.

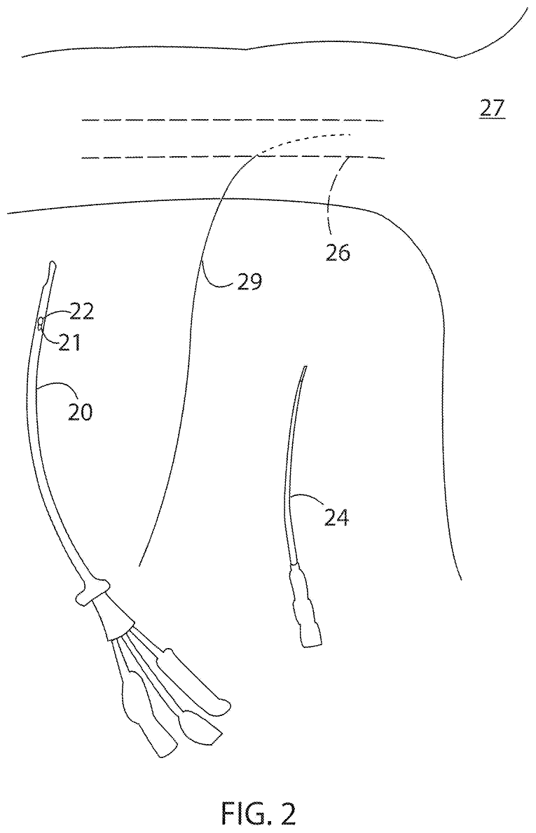

As shown in FIG. 2, a perspective view of another embodiment of the present invention shown in portions near a patient. The view comprises the patient 27 presenting a user (not shown), usually a doctor or nurse, with a vein 26. A short wire 29 has been placed in the vein 26 and the user is ready to insert an improved catheter 20 into the patient's vein. The user would plan to do this through the use of a wire receiving assembly 24 which would be inserted into a hole 22 on the catheter the insertion aided by the deck of an angled portion 21. Though not shown, the short wire 29 would be inserted, slid, into the at least one tube 4 of FIG. 1. The user could then move the catheter 20 along the wire receiving assembly and the wire and into the vein where it is placed.

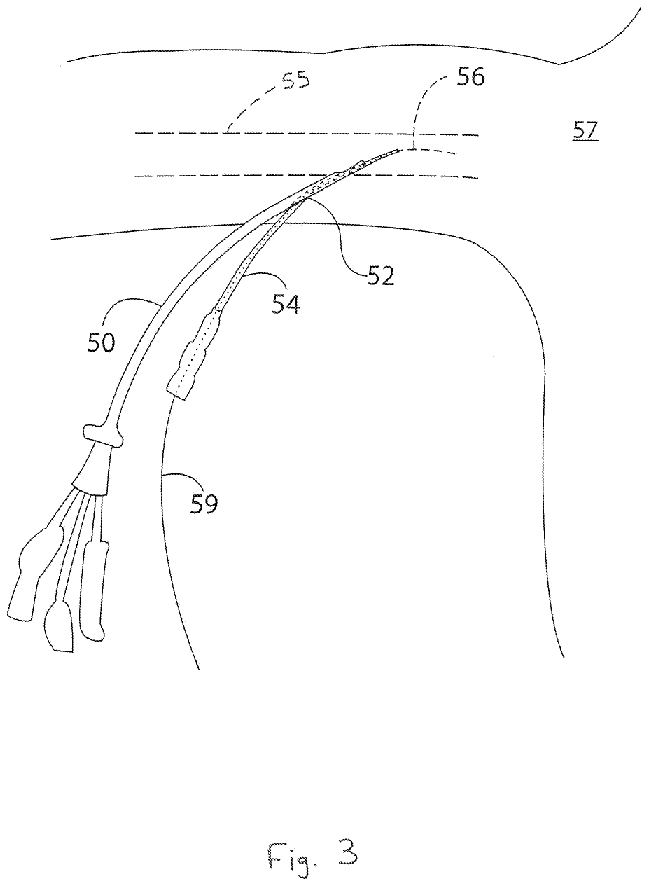

As shown in FIG. 3, a perspective view of another embodiment of the present invention for use with a human body comprises an improved catheter 50. The catheter has at least one hole 52 with a wire receiving assembly 54 inserted. A short wire 56, that was previously placed in the patient's vein 55, is inserted into the first end of the wire assembly 11 of FIG. 1. The first end extended beyond at least one end of the catheter 50.

The improved catheter 50 can be moved toward the patient 57 and guided by the wire receiving assembly 54 and the wire 56 to be inserted into the vein 55 of the patient 57. The wire 59 extends out through the wire receiving assembly.

I found that this catheter design and method of putting the small dilator into the main channel of a catheter via a side hole that extends to the end hole is a versatile design. It can be used to access veins, arteries, abscess, solid organ (liver, kidney) fluid compartments.

My improved method involves using a dilator, a small hollow tube, that then goes into another, slightly larger hollow tube of the catheter. The wire would then go into the dilator at the patient end and be able to go out the distal end of the dilator.

Although the present invention has been described in considerable detail with reference to certain preferred versions thereof, other versions are possible. For instance, the apparatus may incorporate a split tip catheter with multiple side holes or be used on a drainage tube. Therefore, the spirit and scope of the appended claims should not be limited to the description of the preferred versions contained herein.

Any element in a claim that does not explicitly state “means for” performing a specified function, or “step for” performing a specific function, is not to be interpreted as a “means” or “step” clause as specified in 35 U.S.C. § 112, ¶6. In particular, the use of “step of” in the claims herein is not intended to invoke the provisions of 35 U.S.C. § 112, ¶6.

What I claim is:

1. An improved catheter system comprising:
a catheter;
the catheter having at least one first catheter end;
the catheter having an outer surface;
the catheter having at least one hole through the outer surface;
the at least one hole extending to a hollow interior of the catheter;
the at least one first catheter end having an opening;
a wire;
a wire receiving assembly;
the wire receiving assembly having at least one tube;
the wire being slidable within the at least one tube;
the wire receiving assembly having a first end;
the first end being removably inserted into the at least one hole;
the first end being capable of traversing the hollow interior of the catheter;
the first end being passable through the opening and beyond the at least one first catheter end;
the wire being removably inserted into the at least one tube;
the first catheter end being traversable along the first end;
the first catheter end being traversable over the wire.

2. The improved catheter system according to claim 1, wherein the at least one tube is slidably located within the wire receiving assembly.

3. The improved catheter system according to claim 1, wherein the wire is short.

4. The improved catheter system of claim 1 wherein the wire receiving assembly acts as a guide for the at least one first catheter end to slide along and over the wire.

5. The improved catheter system of claim 1 further comprising:
the first end being tapered;
the wire being short;
the catheter having an angled portion formed in the outer surface;
the angled portion being adjacent to at least one of the at least one holes;
the angled portion capable of guiding the first end into the at least one hole;
the at least one hole being located near the at least one first catheter end.

6. The improved catheter system of claim 1 wherein the catheter has a hub end.

7. An improved catheter system comprising:
a catheter;
the catheter having at least one first catheter end;
the catheter having an outer surface;
the catheter having at least one hole through the outer surface;
the at least one hole extending to a hollow interior of the catheter;
the at least one first catheter end having an opening;
a wire;
a wire receiving assembly;
the wire receiving assembly having at least one tube;
the wire being slidable within the at least one tube;
the wire receiving assembly having a first end;
the first end being removably inserted into the at least one hole;
the first end being capable of traversing the hollow interior of the catheter;
the first end being passable through the opening and beyond the at least one first catheter end;
the wire being removably inserted into the at least one tube;
the first catheter end being traversable along the first end;
the first catheter end being traversable over the wire;
the at least one tube being slidably located within the wire receiving assembly;
the wire receiving assembly capable of guiding the at least one first catheter end to slide along and over the wire;
the first end being tapered;
the wire being short;
the catheter having an angled portion formed in the outer surface;
the angled portion being adjacent to at least one of the at least one holes;
the angled portion capable of guiding the first end into the at least one hole;
the at least one hole being located near the at least one first catheter end.

8. The improved catheter system of claim 7 wherein the catheter has a hub end.

9. A method for using an improved catheter system comprising a catheter having at least one first catheter end, the catheter having an outer surface, at least one hole through its outer surface extending to a hollow interior of the catheter, and the at least one first catheter end having an opening, a wire receiving assembly including at least one tube, a wire which is preinserted into a blood vessel and which is insertable into and slidable within the at least one tube, the wire receiving assembly having a first end, wherein the first end is removably inserted into the at least one hole, and the first end is capable of traversing the hollow interior and extending through the opening and beyond the at least one first catheter end, the method comprising the steps of:
inserting the first end into the at least one hole of the catheter;
traversing the hollow interior with the first end;
extending the first end through the opening and beyond the at least one first catheter end;
manipulating the first end such that the first end receives the wire and together the first end and the wire serve to guide the at least one first catheter end over the wire and into the blood vessel;
optionally providing an angled portion formed in the outer surface of the catheter adjacent to at least one of the at least one holes, wherein the angled portion is capable of guiding the first end into the at least one hole, the at least one hole being located near the at least one first catheter end;
optionally providing the catheter with a hub end.

10. A method for using an improved catheter system comprising a catheter having at least one first catheter end, the catheter having an outer surface, at least one hole through its outer surface extending to a hollow interior of the catheter, and the at least one first catheter end having an opening, a wire receiving assembly including at least one tube, a wire which is preinserted into a blood vessel and which is insertable into and slidable within the at least one tube, the wire receiving assembly having a first end, wherein the first end is removably inserted into the at least one hole, and the first end is capable of traversing the hollow interior and extending through the opening and beyond the at least one first catheter end, the method comprising the steps of:
inserting the first end into the at least one hole of the catheter;
traversing the hollow interior with the first end;
extending the first end through the opening and beyond the at least one first catheter end;
manipulating the first end such that the first end receives the wire and together the first end and the wire serve to guide the at least one first catheter end over the wire and into the blood vessel;
providing an angled portion formed in the outer surface of the catheter adjacent to at least one of the at least one holes, wherein the angled portion is capable of guiding the first end into the at least one hole, the at least one hole being located near the at least one first catheter end.
